(19) United States
(12) Patent Application Publication
(10) Pub. No.: US 2005/0038268 A1
(43) Pub. Date: Feb. 17, 2005

(54) Method for the preparation of enterolactone from matairesinol

(76) Inventors: Rainer Sjoholm, Piispanristi (FI); Patrik Eklund, Turku (FI); Anna Lindholm, Bandhagen (SE)

Correspondence Address: JAMES C. LYDON, 100 DAINGERFIELD ROAD, SUITE 100, ALEXANDRIA, VA, 22314, US

(21) Appl. No.: 10/499,482

(22) PCT Filed: Jan. 21, 2003
(86) PCT No.: PCT/FI03/00043

(30) Foreign Application Priority Data
Feb. 5, 2002 (FI) 20020222

(51) Int. Cl. C07D 307/02

(52) U.S. Cl. 549/295

(57) ABSTRACT

This invention concerns a method for the preparation of enterolactone. The method is characterized by the steps of a) transforming the phenolic hydroxyl groups of matairesinol to groups R, which are removable by hydrogenolysis, to give Compound B in Scheme 1, b) substituting the groups R in Compound B in Scheme 1 with hydrogen, to give bis-3,3′-O-methylenterolactone (Compound C), and c) converting the methoxy groups in bis-3,3′-O-methylenterolactone to hydroxyl groups to give enterolactone. The invention concerns also a new group of compounds having the formula of Compound B in Scheme 1.

### FIELD OF THE INVENTION

This invention concerns a method for the synthesis of the mammalian lignan enterolactone from matairesinol.

### BACKGROUND OF THE INVENTION

The publications and other materials used herein to illuminate the background of the invention, and in particular, cases to provide additional details respecting the practice, are incorporated by reference.

Plant lignans such as matairesinol and secoisolariciresinol, are converted by gut microflora to mammalian lignans, enterolactone and enterodiol, correspondingly (Axelson et al., Nature, 298:659-660, 1982).

Enterolactone is known to possess many valuable therapeutical properties. Urinary excretion and serum concentrations of enterolactone are low in women diagnosed with breast cancer (Ingram et al., Lancet, October 4;350 (9083):990-994, 1997; Hultén et al., COST 916 Workshop “Phyto-oestrogens: exposure, bioavailability, health benefits and safety concerns”, 1998) suggesting that this lignan is chemopreventive. The inhibition of aromatase by enterolactone would suggest a mechanism by which consumption of lignan-rich plant food might contribute to reduction of estrogen-dependent diseases, such as breast cancer (Adlercreutz et al., J Steroid Biochem Mol Biol, 44:147-153, 1993; Wang et al., J Steroid Biochem Mol Biol, 50:205-212, 1994). The potential antioxidant activity of enterolactone could also represent a mechanism associated with the preventive action of this lignan in the development of cancers.

The international patent publication WO 00/59946 suggests discloses that hydroxymatairesinol is efficiently converted to enterolactone in vivo and thus useful to increase the level of enterolcatone.

Isolated mammalian lignans such as enterolactone, have, however, not been available earlier in sufficient amounts to be used in animal experiments or clinical trials. The only possibility to increase lignan intake has been to increase the consumption of fiber-rich food items such as flaxseed.

Methods for the synthesis of enterolactone have been disclosed earlier e.g. by M B Groen and J Leemhuis, Tetrahedron Letters 21, 5043 (1980) and G Cooley et al., ibid 22, 349 (1981). The known methods are, however, total syntheses and include at least six steps.

The aim of the present invention is to provide a novel method for the synthesis of large amounts of enterolactone from a plant lignan, which in turn also can be produced in large amounts.

### SUMMARY OF THE INVENTION

This invention concerns a method for the preparation of enterolactone. The method comprises the steps of
a) transformation of the phenolic hydroxyl groups of matairesinol to groups R, which are removable by hydrogenolysis, to give Compound B in Scheme 1,
b) substitution of the groups R in Compound B in Scheme 1 with hydrogen, to give bis-3,3′-O-methylenterolactone, and
c) conversion of the methoxy groups in bis-3,3′-O-methylenterolactone to hydroxyl groups.

### DETAILED DESCRIPTION OF THE INVENTION

The transformation of the phenolic hydroxyl groups in matairesinol (Compound A in Scheme 1) is preferably carried out by reactions leading to derivatives such as esters, ethers, sulfonyl esters, O-arylisoureas, aryl cyanates and aryloxytetrazoles or -benzoxazoles (Compound B in Scheme 1). Preferable reagents are anhydrides or halides of carboxylic acids, phosphoric acids or sulfonic acids as well as carbodiimides, cyanogen bromide, chlorotetrazoles and chlorobenzoxazoles. Particularly preferable are sulfonic acid anhydrides such as triflic acid anhydride.

Compound B in Scheme 1 represents a novel group of compounds.

The substitution of the groups R in Compound B in Scheme 1 with hydrogen atoms by hydrogenolysis is preferably carried out by catalytic hydrogen transfer (homo- or heterogenous conditions) using a hydrogen donor and palladium or Ni complexes, palladium metal on a carrier such as carbon, platinum oxides or Raney-type catalysts such as Raney-Ni. In many cases the hydrogenolysis can be achieved by catalytic hydrogenation. Preferable reagents are hydrogen donors such as acidic trialkylammonium salts, alcohols or metal hydrides together with palladium or Ni complexes as catalysts. Particularly preferable is triethylammonium formate together with PdCl₂(PPh₃)₂ as catalyst and bis(diphenylfosfino)propane as chelating agent.

The resulting compound, bis-3,3′-O-methylenterolactone (Compound C in Scheme 1) has been disclosed previously. Its demethylation to enterolactone (Compound D in Scheme 1) has also been described earlier (Sibi, P. M., Liu, P. and Johnson, M. D., Can J. Chem, 2000, 78, 133; Yoda, H., Kitayama, H., Katagira, T. and Takabe K., Tetrahedron, 1992, Vol. 48, No. 16, 3313.)

The methoxy groups in bis-3,3′-O-methylenterolactone can be converted to hydroxyl groups by several different ether cleavage reactions. Preferably the reaction is carried out by use of Lewis acids, such as boron or aluminium halides, strong mineral acids such as HBr or HI, or metal hydrides, halides, amides, cyanides or sulfides, or silyl halides and silanes. A particularly preferable reagent is BBr₃. Suitable solvents are ethers such as diethyl ether or tetrahydrofuran or halogenated hydrocarbons such as dichloromethane.

According to a preferable alternative, the matairesinol used as starting material is prepared by catalytic hydrogenolysis of the 7-OH-group in hydroxymatairesinol. A method for the preparation of matairesinol from hydroxymatairesinol by use of palladium in acetic acid ester was described by Freudenberg K and Knof L, “Lignanes des Fichtenholzes”. Chem. Ber. 90, 2857-69, 1957. According to novel studies, the method can be essentially improved by using pressurized catalytic hydrogenolysis.

Hydroxymatairesinol can, in turn, be produced in large amounts from wood. It has recently been found that high amounts of hydroxymatairesinol can be produced by extracting finely divided wood material, preferably spruce knotwood, with a polar solvent or solvent mixture and precipitating hydroxymatairesinol from the extract as a complex. Suitable solvents to be used in the extraction step are, for example, pure ethanol or a mixture of ethanol and ethyl acetate. After the extraction step at least part of the solvent is preferably withdrawn before the addition of a complexing agent, which preferable is a carboxylate, such as acetate, of an alkali metal, such as potassium, an earth alkali metal, or ammonium. Such carboxylates form crystallisable adducts with hydroxymatairesinol. An especially preferable complexing agent is potassium acetate, which gives an easily crystallisable potassium acetate adduct of hydroxymatairesinol. This adduct can easily be used as such in the catalytic hydrogenolysis to matairesinol.

The invention is described in more detail by the following non-restrictive Examples.

### EXAMPLE 1

To 1.076 g (3 mmol) of matairesinol (A, see Scheme 1), 2.6 g lutidine and 15 ml of dry dichloromethane were added. The reaction mixture was cooled on an icebath. Under argon 1.2 ml (7.2 mmol) of triflic anhydride was added slowly through a septum. After 68 hours the reaction was ended. 200 ml of dichloromethane was added and the mixture was extracted *5 with distilled water. The organic phase was dried over Na₂SO₄, filtered and the solvent was removed with a rotary evaporator. The product was purified by flash chromatography, (ethylacetate: petoleum ether, 1:3), and 1.640 g (88%) of pure 4,4′-bis-O-trifluoromethane sulfonylmatairesinol (a compound of formula B in Scheme 1) was obtained.

4,4′-Bis-O-trifluoromethanesulfonylmatairesinol:

HRMS m/z calculated for C₂₂H₂₀F₆O₁₀S₂ (M⁺) 622.0402 found 622.0403.

¹H NMR (500 MHz, CDCl₃) δ 2.48(1H, m, H-8′), 2.62(1H, d J=8.7, 6.0 Hz, H-8), 2.69 (1H, dd J=13.4, 7.3 Hz, H-7′), 2.70(1H, dd J=13.6, 7.0 Hz, H-7′), 2.99 (1H, d J=6.7 Hz, H-7), 3.85 (1H, s, CH₃—O′), 3.86 (1H, s, CH₃—O), 3.92 (1H, dd J=9.1, 7.9 Hz, H-9′), 4.23(1H, dd J=9.1, 7.5 Hz, H-9′), 6.60(2H, dd J=8.2, 2.1 Hz, H-6, H-6′), 6.64(1H, d J=2.0 Hz, H-2′), 6.85 (1H, d J=2.0 Hz, H-2), 7.11(1H, d J=8.2 Hz, H-5′), 7.13 (1H, d J=8.2 Hz, H-5). ¹³C NMR (500 MHz, CDCl₃) δ 34.53, 38.49, 40.87, 46.40, 55.18 (CH₃—O′), 55.26 (CH₃—O), 70.96 (C-9′), 113.37, 114.04, 117.47, 118.75 (q, J=332.1 Hz, CF₃), 120.13, 120.64, 121.39, 122.46, 122.82, 139.28, 139.42, 151.62 (2C), 177.81 (C-9).

### EXAMPLE 2

0.622 g (1 mmol) of 4,4′-bis-O-trifluoromethanesulfonylmatairesinol was dissolved in 3 ml of DMF and 0.6 ml triethylamine was added. To the reaction mixture, stirred under argon at 85° C. was added 62 mg (0.15 mmol) of 1,3-bis(diphenylphosphino)propane and 37 mg (0.06 mmol) PdCl₂(PPh₃)₂. Finally formic acid (6 drops) was added. The reaction was ended after 25 hours. 50 ml dichloromethane and 50 ml dist. water were addded. The organic phase was washed with 6*30 ml 10% HCl solution, 30 ml Brine and dried over Na₂SO₄, filtered and finally the solvent was removed under reduced pressure. The reaction mixture was filtered though a 3 cm layer of silica, and gave 0.73 mmol, 0.237 g (73%) of bis-3,3′-O-methylenterolactone (C) in 90% purity.

### EXAMPLE 3

80% pure C (estim. 0.67 mmol) was dissolved in 3 ml of dichloromethane, the reaction mixture was cooled to −79° C. before 1.6 ml of 1M BBr₃ in diethyl ether was slowly added. After 2 hours of reaction at −78° C. the mixture was allowed to warm to room temperature over night. The following day the mixture was cooled again and 1 ml of methanol was added. Later 30 ml of NaHCO₃ solution and 50 ml ethylacetate were added. The pH value was adjusted to 6-7 with dilute HCl solution, and the organic phase was washed with brine (2*50 ml) and finally dried over NaₛSO₄.

The demethylation was successful and after the reaction no C could be found. According to GC analysis the yield of enterolactone was 60%.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

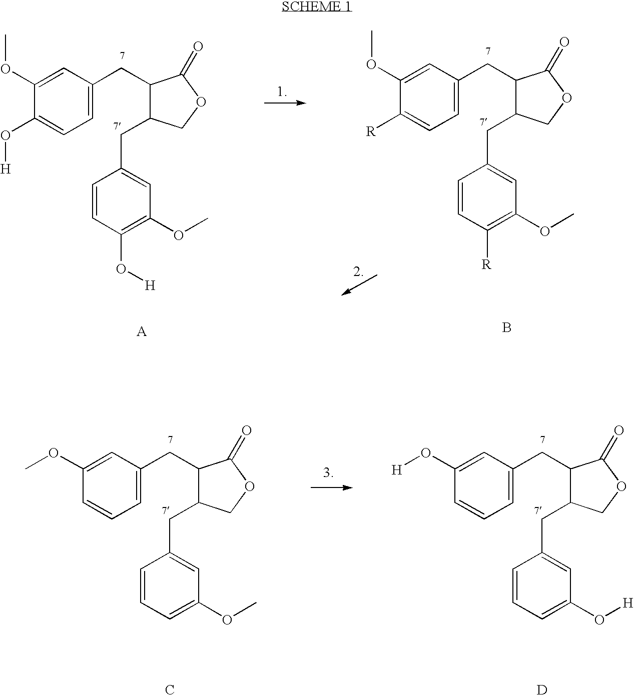

1-12. (Canceled)

13. Method for the preparation of enterolactone from matairesinol, comprising the steps of
a) transforming the phenolic hydroxyl groups of matairesinol to groups R, which are removable by hydrogenolysis, to give Compound B in Scheme 1,
b) substituting the groups R in Compound B in Scheme 1 with hydrogen, to give bis-3,3′-O-methylenterolactone, and
c) converting the methoxy groups in bis-3,3′-O-methylenterolactone to hydroxyl groups to give enterolactone.

14. The method of claim 13, wherein the phenolic hydroxyl groups of matairesinol are transformed to such R-groups, which define the Compound B in Scheme 1 as an ester, ether, sulfonyl ester, O-arylisourea, aryl cyanate, aryloxytetrazole or arylbenzoxazole.

15. The method of claim 14, wherein the transformation step is carried out by use of an anhydride or halide of a carboxylic acid, phosphoric acid, or sulfonic acid; a carbodiimide; cyanogen bromide; a chlorotetrazole or a chlorobenzoxazole.

16. The method of claim 15, wherein the transformation step is carried out by use of a sulfonic acid anhydride.

17. The method of claim 16, wherein the sulfonic acid anhydride is triflic acid anhydride.

18. The method of claim 13, wherein the substitution of the groups R in Compound B in Scheme 1 with hydrogen atoms is carried out by catalytic hydrogen transfer using a hydrogen donor and catalyst which is a palladium or Ni complex, palladium metal on a carrier such as carbon, platinum oxide or a Raney-type catalyst such as Raney-Ni.

19. The method of claim 13, wherein the substitution of the groups R in Compound B in Scheme 1 with hydrogen atoms is carried out by catalytic hydrogenation.

20. The method of claim 18, wherein the substitution reaction is carried out by use of a hydrogen donor such as hydrogen, an acidic trialkylammonium salt, an alcohol or a metal hydride together with a palladium or Ni complex as catalysts.

21. The method of claim 20, wherein the reaction is carried out by use of triethylammonium formate together with PdCl₂(PPh₃)₂ as catalyst and bis(diphenylfosfino)propane as chelating agent.

22. A compound having a formula of Compound B as disclosed in Scheme 1, wherein the R-groups have such meanings that said compound is an ester, ether, sulfonyl ester, O-arylisourea, aryl cyanate, aryloxytetrazole or arylbenzoxazole.

23. The compound of claim 22, wherein R is a sulfonyloxy group.

24. The compound of claim 23, wherein the sulfonyloxy group is trifluoromethane sulfonyloxy.
